(19) United States
(12) Patent Application Publication
(10) Pub. No.: US 2026/0118346 A1
(43) Pub. Date: Apr. 30, 2026

(54) METHODS AND RELATED ASPECTS FOR DETERMINING COGNITIVE STATUS ASSOCIATED WITH PARKINSON'S DISEASE

(71) Applicant: THE JOHNS HOPKINS UNIVERSITY, Baltimore, MD (US)

(72) Inventors: Xiaobo MAO, New York, NY (US); Liana ROSENTHAL, Ellicott City, MD (US); Ted M. DAWSON, Baltimore, MD (US); Valina L. DAWSON, Baltimore, MD (US)

(21) Appl. No.: 19/163,548

(22) PCT Filed: Oct. 31, 2024
(86) PCT No.: PCT/US2024/053963

§ 371 Date: Sep. 9, 2025

Related U.S. Application Data
(60) Provisional application No. 63/595,168, filed on Nov. 1, 2023.

(51) Int. Cl. G01N 33/50

(52) U.S. Cl. CPC G01N 33/5058; G01N 33/5091

(57) ABSTRACT

Provided herein are methods of determining a cognitive status of a test subject. In some embodiments, the methods include passing a test subject data set through a model that relates test subject data sets to cognitive status of the test subjects in which the model was created using reference subject data sets produced by determining three or more features from samples obtained from the reference subjects using at least a thioflavin T (ThT) assay, a dynamic light scattering (DLS) assay, and a neurotoxicity assay. In some embodiments, the methods also include outputting from the model a classification of the test subject as having a Parkinson's disease (PD) status of PD-NC (normal cognition) or PD-CI (cognitive impairment). Related systems, computer readable media, and additional methods are also provided.

### CROSS-REFERENCE TO RELATED APPLICATIONS

This application is the national stage entry of International Patent Application No. PCT/US2024/053963, filed on Oct. 31, 2024, and published as WO 2025/096829 A1 on May 8, 2025, which claims the benefit of U.S. Provisional Patent Application Ser. No. 63/595,168, filed Nov. 1, 2023, which are hereby incorporated by reference herein in their entireties.

### STATEMENT REGARDING FEDERALLY SPONSORED RESEARCH OR DEVELOPMENT

This invention was made with Government support under award nos. AG056841, NS125559 and NS125592, awarded by the National Institutes of Health. The Government has certain rights in the invention.

### FIELD

This disclosure relates generally to machine learning, e.g., in the context of medical applications, such as diagnostics and pharmacology.

### BACKGROUND

Misfolded α-synuclein (α-syn) is a key pathological feature underlying the motor and cognitive changes in individuals with Parkinson's disease (PD). Seed amplification assays (SAAs) of α-syn have been well-characterized as potential diagnostic biomarkers for PD, including the discrimination of PD from multiple system atrophy (MSA). There's an urgent need for progression biomarkers directly related to PD-pathophysiology to enhance patient care, facilitate clinical trial cohort selection, and aid in the development of new therapeutics.

To that end, SAAs can also be used to evaluate various aggregation properties of α-syn strains and amplify α-syn strains from patients at different stages of their respective α-synucleinopathies. This enables researchers to characterize strain properties associated with or result in differential disease progression.

Disease progression in PD includes greater motor impairment over time coupled with the growth of non-motor symptoms, including cognitive change. Patients initially demonstrate limited to no cognitive changes early in the disease (PD-NC) and later develop cognitive impairment (PD-CI), including PD-MCI (mild CI) and then PD-D (dementia). The rate and severity of this cognitive change are highly variable between patients, which has been attributed to the presence of other proteinopathies at autopsy and the extent of α-syn cortical burden.

Accordingly, there is a need for additional methods of determining or predicting cognitive status associated with Parkinson's disease.

### SUMMARY

The present disclosure provides, in certain aspects, an artificial intelligence (AI) system capable of determining the cognitive status of test subjects, for example, as part of a Parkinson's disease diagnosis or evaluation. These and other aspects will be apparent upon a complete review of the present disclosure, including the accompanying figures.

According to various embodiments, a method of determining a cognitive status of a test subject is presented. The method includes determining three or more features from a sample obtained from the test subject using at least a thioflavin T (ThT) assay, a dynamic light scattering (DLS) assay, and a neurotoxicity assay to produce a test subject data set; and using the test subject data set to classify the test subject as having a Parkinson's disease (PD) status of PD-NC (normal cognition) or PD-CI (cognitive impairment), thereby determining the cognitive status of the test subject.

Various optional features of the above embodiments include the following. The method comprises determining four or more, five or more, six or more, seven or more, eight or more, nine or more, ten or more, or more features from the sample. The features determined from the ThT assay comprise one or more of a ThT-mfi value, a ThT-t_{lag} value, or a ThT-t₅₀ value. The features determined from the DLS assay comprise one or more of a peak number value, a size of peak ½ value, an intensity of peak ½ value. When the cognitive status of the test subject is classified as PD-CI, the method further comprises classify the test subject as having PD-MCI (mild cognitive impairment) or PD-D (dementia). The method further comprises repeating the method one or more times using samples obtained from the test subject at subsequent time points to evaluate the cognitive status of the test subject over a selected period of time. The method further comprises predicting a probable disease progression outcome for the test subject based at least in part on the cognitive status of the test subject. The method further comprises administering or discontinuing administering a therapy to the test subject based at least in part on the cognitive status of the test subject. The method further comprises generating a therapy recommendation for the test subject based at least in part on the cognitive status of the test subject.

According to various embodiments, a computer-implemented method of determining a cognitive status of a test subject is presented. The method includes passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and, outputting from the model a classification of the test subject as having a Parkinson's disease (PD) status of PD-NC (normal cognition) or PD-CI (cognitive impairment), thereby predicting the determining the cognitive status of the test subject.

Various optional features of the above embodiments include the following. The model was created using the reference subject data sets produced by determining four or more, five or more, six or more, seven or more, eight or more, nine or more, ten or more, or more features from samples obtained from the reference subjects. The features determined from the ThT assay comprise one or more of a ThT-mfi value, a ThT-t_{lag} value, or a ThT-t₅₀ value. When the cognitive status of the test subject is classified as PD-CI, the model further outputs a classification of the test subject as having PD-MCI (mild cognitive impairment) or PD-D (dementia). The method further comprises outputting from the model a prediction of a probable disease progression outcome for the test subject based at least in part on the cognitive status of the test subject. The method further comprises outputting from the model a therapy recommendation for the test subject based at least in part on the cognitive status of the test subject. A trained electronic neural network comprises the model. The method comprises determining three or more features from a sample obtained from the test subject using at least a thioflavin T (ThT) assay, a dynamic light scattering (DLS) assay, and a neurotoxicity assay to produce the test subject data set. The method comprises determining four or more, five or more, six or more, seven or more, eight or more, nine or more, ten or more, or more features from the sample obtained from the test subject. The features determined from the ThT assay comprise one or more of a ThT-mfi value, a ThT-t_{lag} value, or a ThT-t₅₀ value. The features determined from the DLS assay comprise one or more of a peak number value, a size of peak ½ value, an intensity of peak ½ value.

According to various embodiments, a system for determining a cognitive status of a test subject is presented. The system comprising: an analytical component that is capable of receiving a sample obtained from the test subject, which analytical component is configured to determine three or more features from the sample obtained from the test subject using at least a thioflavin T (ThT) assay, a dynamic light scattering (DLS) assay, and a neurotoxicity assay to produce a test subject data set when the analytical component receives the sample; and, a controller that is operably connected, or connectable, at least to the analytical component, wherein the controller comprises, or is capable of accessing, computer readable media comprising non-transitory computer executable instructions which, when executed by at least one electronic processor, perform at least: using the test subject data set to classify the test subject as having a Parkinson's disease (PD) status of PD-NC (normal cognition) or PD-CI (cognitive impairment).

According to various embodiments, a system for determining a cognitive status of a test subject, the system comprising: a processor; and a memory communicatively coupled to the processor, the memory storing instructions which, when executed on the processor, perform operations comprising: passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and, outputting from the model a classification of the test subject as having a Parkinson's disease (PD) status of PD-NC (normal cognition) or PD-CI (cognitive impairment).

According to various embodiments, a computer readable media is presented. The computer readable media comprising non-transitory computer executable instructions which, when executed by at least one electronic processor, perform at least: receiving a test subject data set comprising three or more features determined from a sample obtained from the test subject using at least a thioflavin T (ThT) assay, a dynamic light scattering (DLS) assay, and a neurotoxicity assay; and, using the test subject data set to classify the test subject as having a Parkinson's disease (PD) status of PD-NC (normal cognition) or PD-CI (cognitive impairment).

According to various embodiments, a computer readable media is presented. The computer readable media comprising non-transitory computer executable instructions which, when executed by at least one electronic processor, perform at least: passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and, outputting from the model a classification of the test subject as having a Parkinson's disease (PD) status of PD-NC (normal cognition) or PD-CI (cognitive impairment).

### DRAWINGS

The above and/or other aspects and advantages will become more apparent and more readily appreciated from the following detailed description of examples, taken in conjunction with the accompanying drawings, in which:

FIG. 1A is a flow chart that schematically shows exemplary method steps of determining a cognitive status of a test subject according to some aspects disclosed herein.

FIG. 1B is a flow chart that schematically shows exemplary method steps determining a cognitive status of a test subject according to some aspects disclosed herein.

FIG. 1C is a schematic diagram of an exemplary system suitable for use with certain aspects disclosed herein.

FIG. 1D. α-Syn strain changes allied with cognitive changes in Parkinson's disease. Cross-sectional and longitudinal studies show the features of α-syn strains change with and before the diagnosis of the cognitive decline in PD, with Thioflavin T (ThT) assay, Dynamic light scattering (DLS) assay, and neurotoxicity assay. Combined features of α-syn strains can better predict PD subtypes and cognitive status.

FIGS. 2A-2G. Differentiating amplified α-syn aggregates derived from patients with PD-NC, PD-MCI, and PD-D using Thioflavin T (ThT) assay. (a) Schematic representation of α-syn amplification and characterization using ThT assay. In these groups: HC (health control), PD-NC (normal cognition), PD-MCI (mild cognitive impairment), PD-D (dementia), and CSF samples were amplified with SAA and ThT assay were performed. (b) ThT-mfi (maximal fluorescence intensity) of CSF-SAA samples in Cohort I. HC (n=24), PD-NC (n=24), PD-MCI (n=28), and PD-D (n=8). (c) ThT-t_{lag} (time at which aggregation started) of CSF-SAA samples in Cohort I. PD-NC (n=22), PD-MCI (n=28), and PD-D (n=8). (d) ThT-t₅₀ (time at which aggregation completed 50%) of CSF-SAA samples in Cohort I. PD-NC (n=22), PD-MCI (n=28) and PD-D (n=8). (e) ThT-mfi of CSF-SAA samples in Cohort II. PD-NC (n=34), PD-MCI (n=47) and PD-D (n=7). (f) ThT-t_{lag} of CSF-SAA samples in Cohort II. PD-NC (n=32), PD-MCI (n=46) and PD-D (n=7). (g) ThT-t₅₀ of CSF-SAA samples in Cohort II. Data are mean±SEM. The statistical significance was evaluated via one-way ANOVA with Tukey's multiple comparisons test. ****P<0.0001. Every dot indicates an individual biological sample measured in duplicate.

FIGS. 3A-3K. Differentiating α-syn strains using dynamic light scattering (DLS), cell-based and biochemical assays. (a) Schematic representation of the characterization of α-syn strains from HC, PD-NC, PD-MCI, and PD-D. DLS data processing provides peak number, peak size, and peak intensities. Neuron culture study provides neurotoxicity results. (b) The number of DLS peaks in Cohort I for HC (n=24), PD-NC (n=24), PD-MCI (n=28) and PD-D (n=8). (c) The number of DLS peaks in Cohort II for PD-NC (n=34), PD-MCI (n=37), and PD-D (n=7). (d) DLS spectra of amplified α-syn strains from HC, PD-NC, PD-MCI, and PD-D. (e & f) Neurotoxicity of α-syn strains assessed with the immunostaining of anti-NeuN (neuronal nuclei marker) in Cohort I, and quantification. Scale bar, 50 μm. Cohort I: HC (n=24), PD-NC (n=24), PD-MCI (n=28), and PD-D (n=8). (g) Neurotoxicity of α-syn strains in Cohort II. (h & i) Dot-blot representing proteinase K (PK)-digested α-syn strains and quantification. (j & k) SDS-PAGE followed by silver staining representing PK-digested α-syn strains and quantification. (b, c, f, g, i, k) Each dot represents an individual biological sample. The violin plot shows all the points. Data are presented as the mean±SEM. The statistical significance was evaluated via one-way ANOVA with Tukey's multiple comparisons test. No significant difference (ns) P>0.05, *P<0.05, **P<0.01, ***P<0.001, ****P<0.0001.

FIGS. 4A-4C. Artificial Intelligence (AI) predicted disease using ThT, DLS, and neurotoxicity data. (a) Schematic representation of how AI was used to predict disease using ThT, DLS, and neurotoxicity. Table for prediction of disease by AI six model (ET, GBDT, RF, CB, DT, XGB) in the test data set using all features data. The importances of nine features in the ET model was shown in the right panel. (b) The prediction accuracy by AI ET model using 9 features.

FIGS. 5A-5G. Longitudinal analysis for α-syn strains using DLS. (a) Schematic representation for DLS analysis of amplified α-syn strains from longitudinal HC, PD-NC, PD-MCI, and PD-D groups. (b) Nasted plot for the peak number between the first- and last-visit in Cohort I. Different colors indicate the different stages of cognitive status. (c,d,f,g) Yearly mapping of the DLS peak number of amplified α-syn strains from individuals with stable cognitive status. Black: HC; blue: PD-NC; green: PD-MCI; red: PD-D. (e) Yearly mapping of the DLS peak number of amplified α-syn strains from individuals with changed cognitive status. The statistical significance was evaluated via students t-test. No significant difference (n.s) P>0.05, ****P<0.0001.

FIGS. 6A and 6B. Longitudinal prediction using AI. (a) Schematic representation for survival analysis using AI. (b) table shows the combination of features and c-index values for different survival analysis models predicting cognitive impairment.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

α-synuclein: As used herein, the term “α-synuclein,” “α-syn,” “alpha-synuclein,” or “SNCA” refers to a protein member of the synuclein family, which also includes beta- and gamma-synuclein. Synucleins are abundantly expressed in the brain and alpha- and beta-synuclein inhibit phospholipase D2 selectively. SNCA may serve to integrate presynaptic signaling and membrane trafficking. Defects in SNCA have been implicated in the pathogenesis of, for example, Parkinson disease. SNCA peptides are also a major component of amyloid plaques in the brains of patients with Alzheimer's disease. Alternatively spliced transcripts encoding different isoforms have been identified for this gene. A human α-synuclein NCBI Gene ID No. is 6622. α-synuclein can be present in various forms, such as monomeric α-syn, oligomeric α-syn, and preformed-fibrillar (PFF) α-syn.

Data set. As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Prion-like Protein: As used herein, “prion like protein” refers to a neurodegenerative disease-related protein that shares similarities with prion replication and propagation processes, but which has noninfectious characteristics, unlike a prion. Examples of prion-like proteins, include amyloid-β (Aβ), α-synuclein, tau, and the transactive response DNA-binding protein of 43 kDa (TDP-43).

Protein: As used herein, “protein” is used interchangeably with “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties. The term “polypeptide” typically refers to a sequence with more than 10 amino acids and the term “peptide” means sequences with up to 10 amino acids in length. However, the terms may be used interchangeably.

Proteinopathy: As used herein, “proteinopathy” or “protein conformational disorder,” or “protein misfolding disease,” is a class of diseases in which certain proteins become structurally abnormal, and thereby disrupt the function of cells, tissues and organs of the body. Frequently, the proteins fail to fold into their normal configuration; in this misfolded state, the proteins become toxic in some way (e.g., a toxic gain-of-function) or they lose their normal function. Examples of proteinopathies include diseases such as Creutzfeldt-Jakob disease and other prion diseases, Alzheimer's disease, Parkinson's disease, Lewy body dementia (LBD), amyloidosis, multiple system atrophy, and a wide range of other neurodegenerative disorders. In some embodiments, the proteinopathy is “α-synucleinopathy” which are a class of diseases involving misfolded prion-like neuronal protein α-synuclein. Other examples of prion-like proteins, include amyloid-β (Aβ), tau, and the transactive response DNA-binding protein of 43 kDa (TDP-43).

Sample: As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. In a particular example, a sample includes blood obtained from a subject, such as whole blood or serum. In another example, a sample includes cells collected using an oral rinse. The sample may be isolated from the subject and then directly utilized in a method for determining the presence or absence of antibodies, or alternatively, the sample may be isolated and then stored (e.g., frozen) for a period of time before being subjected to analysis.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

System: As used herein, “system” in the context of analytical instrumentation refers a group of objects and/or devices that form a network for performing a desired objective.

Value: As used herein, “value” generally refers to an entry in a data set that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. Introduction

α-Synuclein (α-syn) strains can serve as discriminators of Parkinson's disease (PD) from other α-synucleinopathies. However, the relationship between α-syn strain dynamics and clinical performance remains unclear. In aspects of the present disclosure, cerebrospinal fluid samples from two PD cohorts were used to amplify α-syn and characterize the amplified strains through various assays. PD patients were categorized into three groups based on cognitive status: PD-NC (normal cognition), PD-CI (including PD-MCI (mild cognitive impairment) and PD-D (dementia)). The results presented herein demonstrate the specificity of α-syn strains in relation to cognitive changes. Leveraging artificial intelligence (AI), in some embodiments, the present disclosure employs machine learning classifiers to achieve high accuracy rates in different classification tasks. The combination of multiple features for model training yielded superior performance (95˜99% accuracy in the 4- and 2-classification) compared to individual features alone. Longitudinal studies revealed α-syn strain changes preceding the diagnosis of cognitive decline, particularly from PD-NC to PD-MCI. Utilizing a machine learning survival analysis model as described herein, the inventors identified ThT-mfi and ThT-t_{lag} features as significantly impacting cognitive changes. Both cross-sectional and longitudinal analyses highlight the distinct α-syn strains in PD individuals at different cognitive stages, with strain features correlated to cognitive status. The present disclosure underscores the utility of α-syn strain dynamics as a biomarker for PD and PD-CI. These and other attributes of the present disclosure will be apparent upon a complete review of this specification, including accompanying figures.

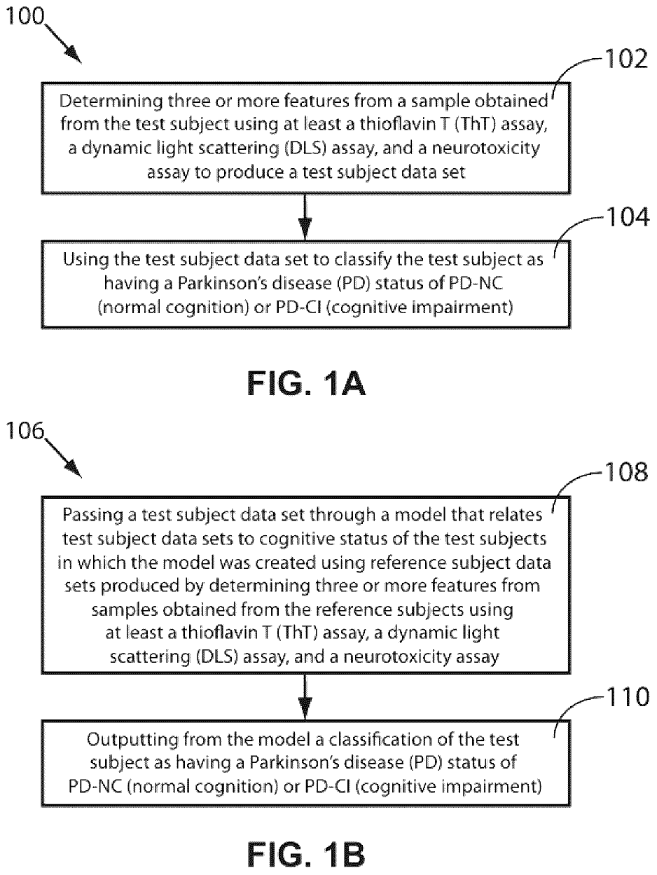

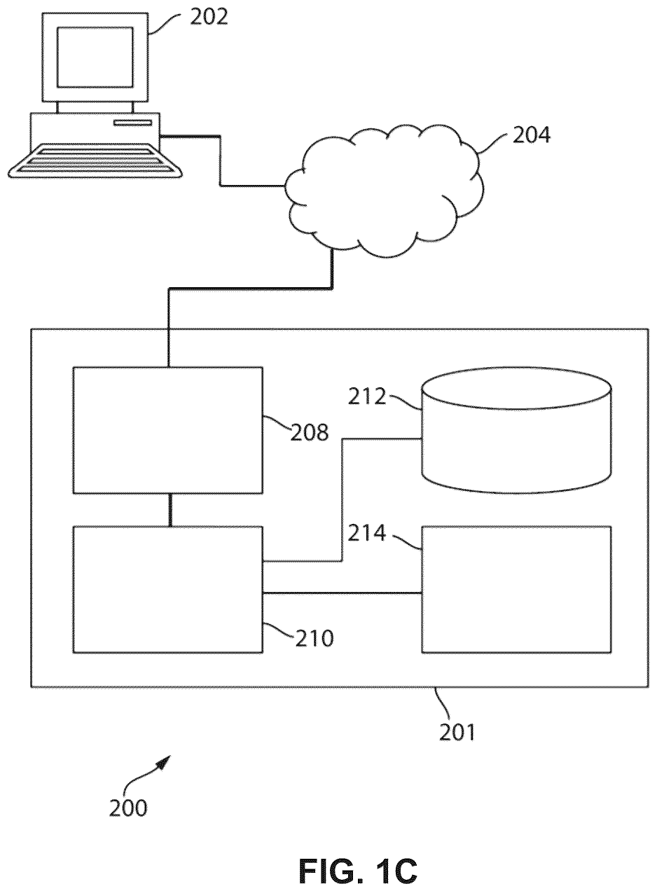

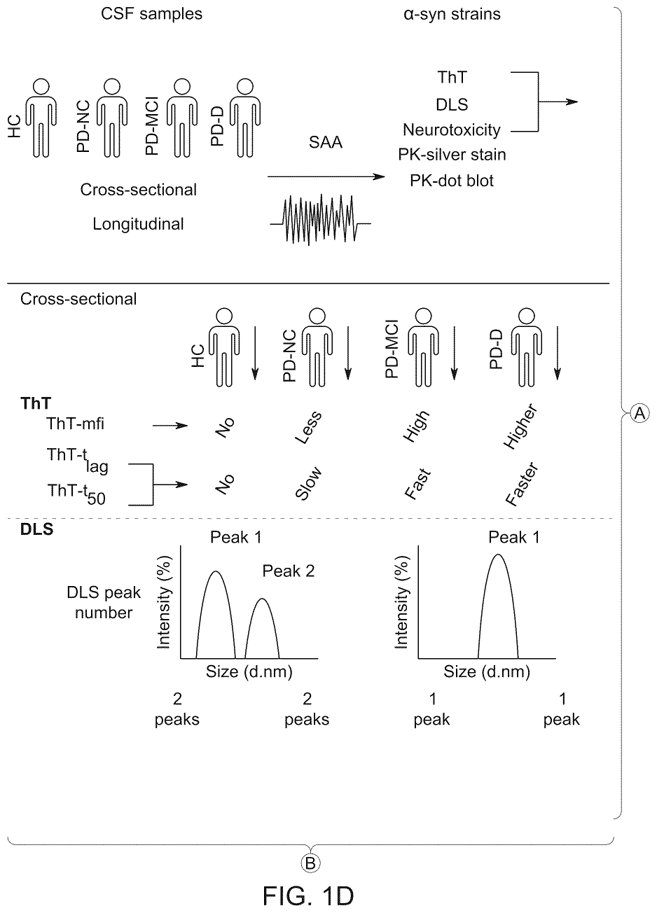

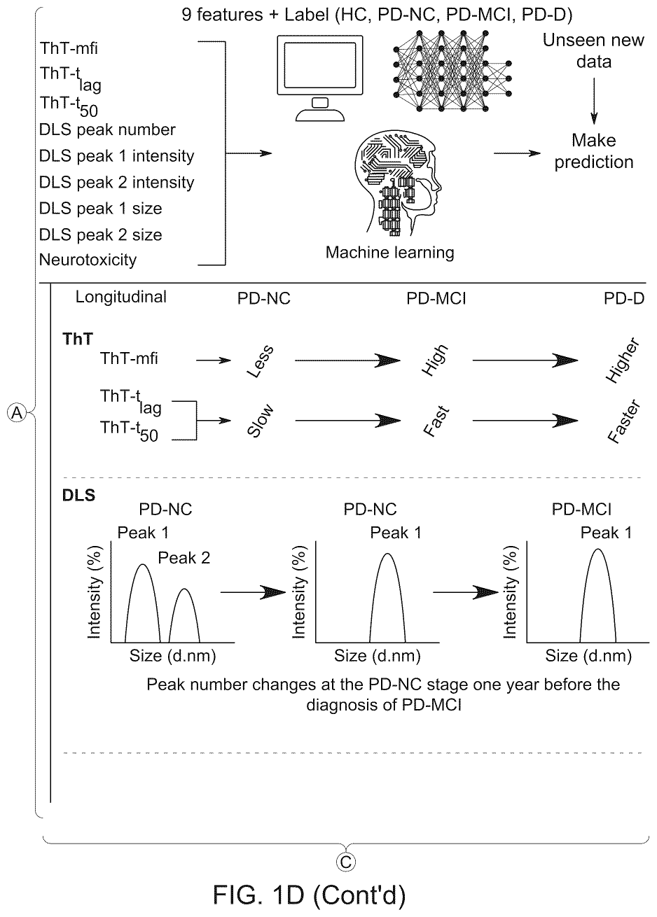

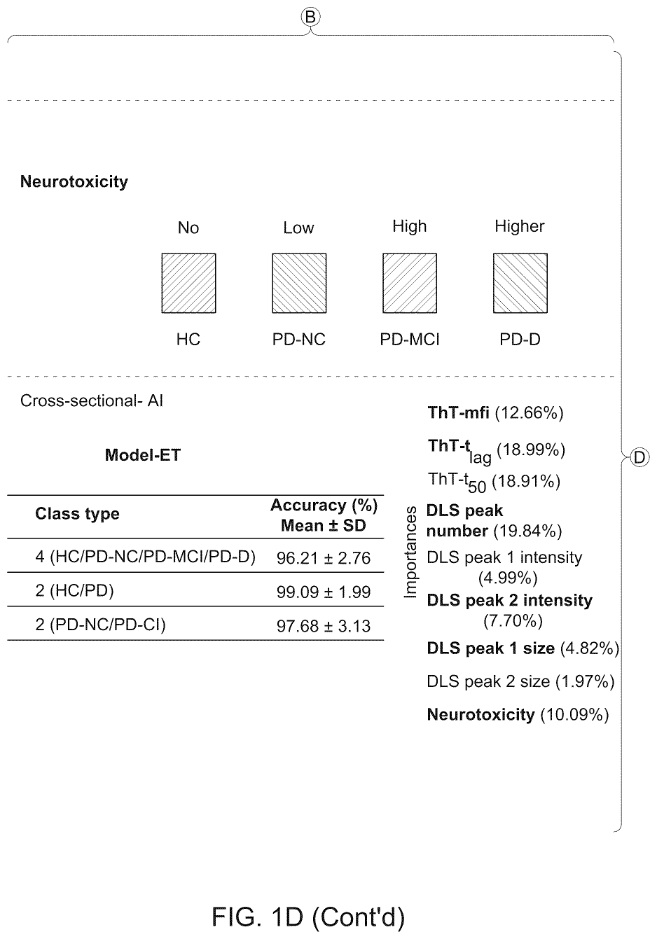

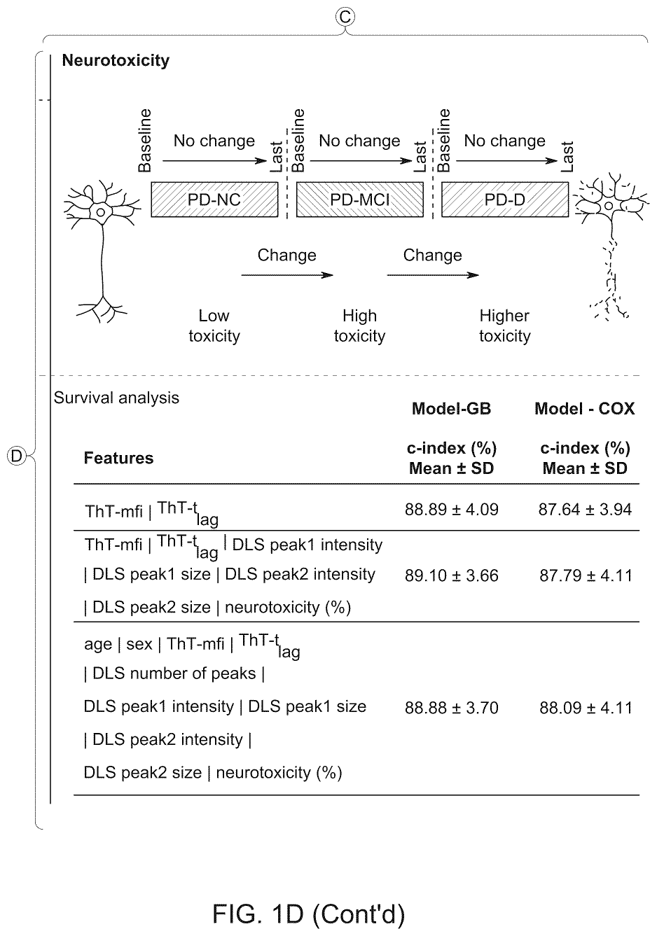

To illustrate, FIG. 1A is a flow chart that schematically shows exemplary method steps of determining a cognitive status of a test subject according to some aspects disclosed herein. As shown, method 100 includes determining three or more features from a sample obtained from the test subject using at least a thioflavin T (ThT) assay, a dynamic light scattering (DLS) assay, and a neurotoxicity assay to produce a test subject data set (step 102). Method 100 also includes using the test subject data set to classify the test subject as having a Parkinson's disease (PD) status of PD-NC (normal cognition) or PD-CI (cognitive impairment) to thereby determining the cognitive status of the test subject. (step 104).

In some embodiments, method 100 includes determining four or more, five or more, six or more, seven or more, eight or more, nine or more, ten or more, or more features from the sample. In some embodiments, the features determined from the ThT assay comprise one or more of a ThT-mfi value, a ThT-t_{lag} value, or a ThT-t₅₀ value. In some embodiments, the features determined from the DLS assay comprise one or more of a peak number value, a size of peak ½ value, an intensity of peak ½ value. In some embodiments, when the cognitive status of the test subject is classified as PD-CI, the method further comprises classify the test subject as having PD-MCI (mild cognitive impairment) or PD-D (dementia). In some embodiments, method 100 further includes repeating the method one or more times using samples obtained from the test subject at subsequent time points to evaluate the cognitive status of the test subject over a selected period of time. In some embodiments, method 100 further includes predicting a probable disease progression outcome for the test subject based at least in part on the cognitive status of the test subject. In some embodiments, method 100 further includes administering or discontinuing administering a therapy to the test subject based at least in part on the cognitive status of the test subject. In some embodiments, method 100 further includes generating a therapy recommendation for the test subject based at least in part on the cognitive status of the test subject.

To further illustrate aspects of the present disclosure, FIG. 1B is a flow chart that schematically shows exemplary computer-implemented method steps of determining a cognitive status of a test subject according to some aspects disclosed herein. As shown, method 106 includes passing at least one test subject data set through a model that relates test subject data sets to cognitive status of the test subjects in which the model was created using one or more reference subject data sets produced by determining three or more features from samples obtained from the reference subjects using at least a thioflavin T (ThT) assay, a dynamic light scattering (DLS) assay, and a neurotoxicity assay (step 108). In addition, method 106 also includes outputting from the model a classification of the test subject as having a Parkinson's disease (PD) status of PD-NC (normal cognition) or PD-CI (cognitive impairment), thereby predicting the determining the cognitive status of the test subject (step 110).

In some embodiments, the model was created using the reference subject data sets produced by determining four or more, five or more, six or more, seven or more, eight or more, nine or more, ten or more, or more features from samples obtained from the reference subjects. In some embodiments, the features determined from the ThT assay comprise one or more of a ThT-mfi value, a ThT-t_{lag} value, or a ThT-t₅₀ value. In some embodiments, when the cognitive status of the test subject is classified as PD-CI, the model further outputs a classification of the test subject as having PD-MCI (mild cognitive impairment) or PD-D (dementia). In some embodiments, method 106 further includes outputting from the model a prediction of a probable disease progression outcome for the test subject based at least in part on the cognitive status of the test subject. In some embodiments, method 106 further includes outputting from the model a therapy recommendation for the test subject based at least in part on the cognitive status of the test subject. In some embodiments, a trained electronic neural network comprises the model. In some embodiments, method 106 includes determining three or more features from a sample obtained from the test subject using at least a thioflavin T (ThT) assay, a dynamic light scattering (DLS) assay, and a neurotoxicity assay to produce the test subject data set. In some embodiments, method 106 includes determining four or more, five or more, six or more, seven or more, eight or more, nine or more, ten or more, or more features from the sample obtained from the test subject. In some embodiments, the features determined from the ThT assay comprise one or more of a ThT-mfi value, a ThT-t_{lag} value, or a ThT-t₅₀ value. In some embodiments, the features determined from the DLS assay comprise one or more of a peak number value, a size of peak ½ value, an intensity of peak ½ value.

FIG. 1C is a schematic diagram of a hardware computer system 200 suitable for implementing various embodiments. For example, FIG. 1C illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic records system, such as an LIS, a database, a compendium of clinical data, or any other source of test and/or reference subject data sets suitable for use as a training corpus as disclosed herein. According to some embodiments, each component is implemented as a vector, such as a feature vector.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. Description of Example Embodiments

Example: Combined Features of Dynamic α-Synuclein Strains Correlated with Cognitive Changes in Parkinson's Disease

### Introduction

We theorized that because α-syn strains underlie the heterogeneity of α-synucleinopathies, it is possible that there are multiple α-syn strains in different stages of PD. Further, α-syn aggregation properties have thus far been considered to be stable in each individual. We hypothesize that α-syn strain dynamics differ amongst individuals and within an individual over time and can therefore be used as a biomarker reflecting cognitive disease progression. To investigate this hypothesis, we collected cerebrospinal fluid (CSF) samples from well-characterized patients from two independent cohorts. We then determined the biophysical, biochemical, and cellular characteristics of the amplified α-syn aggregates and evaluated differences in these properties as they relate to the patient's clinical characteristics in a cross-sectional and longitudinal manner.

### α-Syn Strain Properties Relate to Cognition but not Other Clinical Characteristics

We first sought to evaluate the relationship between the different properties of α-syn aggregation and the clinical characteristics of Cohort I (Johns Hopkins cohort) at baseline. We determined that cognitive status (as determined by consensus conference diagnosis and stratified into PD-NC and PD-CI, see methods below) was the only clinical variable that significantly predicted α-syn strain toxicity. We, therefore, focused on cognition as the outcome measure for subsequent analyses and sought to determine whether α-syn aggregation properties were different amongst different cognitive strata and if these properties predicted current and then future cognitive status. To further enhance our findings, we also looked at motor symptoms (as defined by the Movement Disorder Society-Unified Parkinson's Disease Rating Scale (MDS-UPDRS)) as the outcome variable in both our cross-sectional and prediction models and found that α-syn strain properties do not relate to motor symptomatology.

Cross-Sectional: Clinical Correlation of Amplified α-Syn Strains from CSF of PD Patients

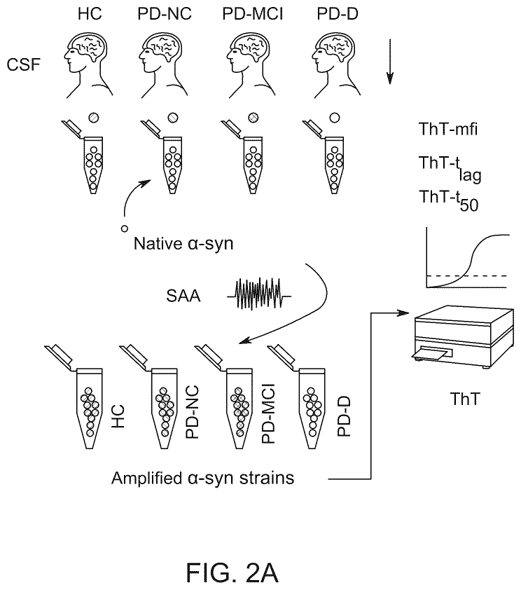

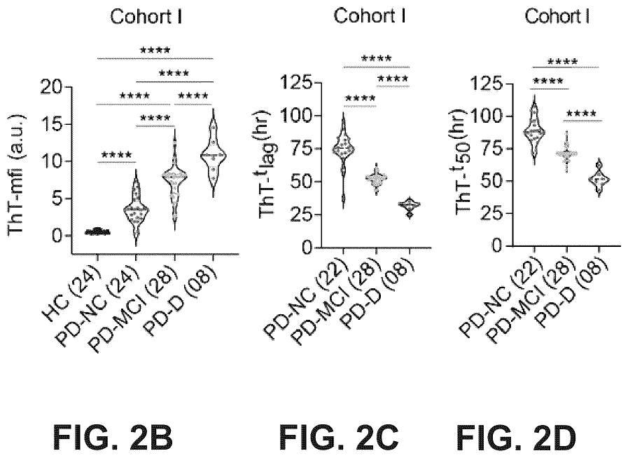

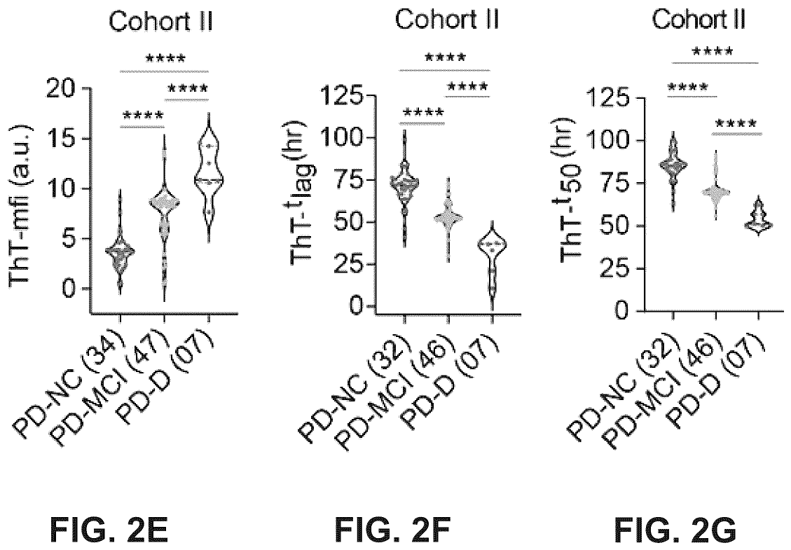

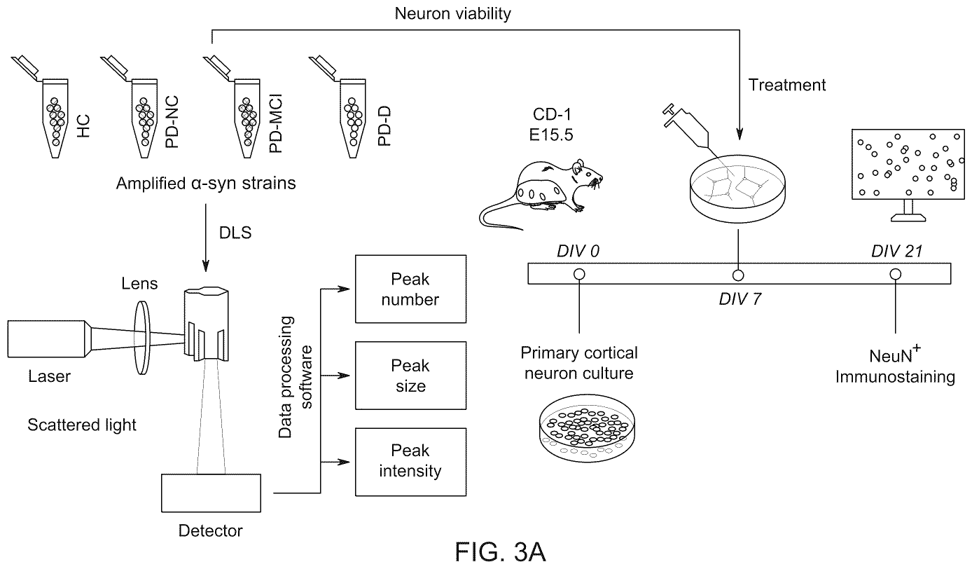

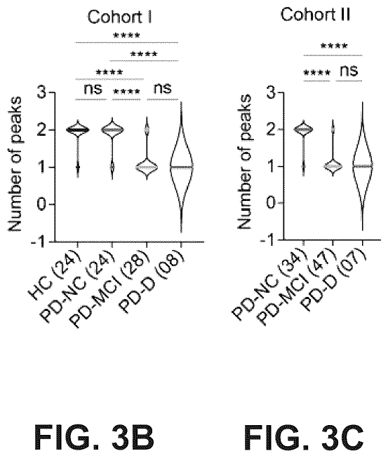

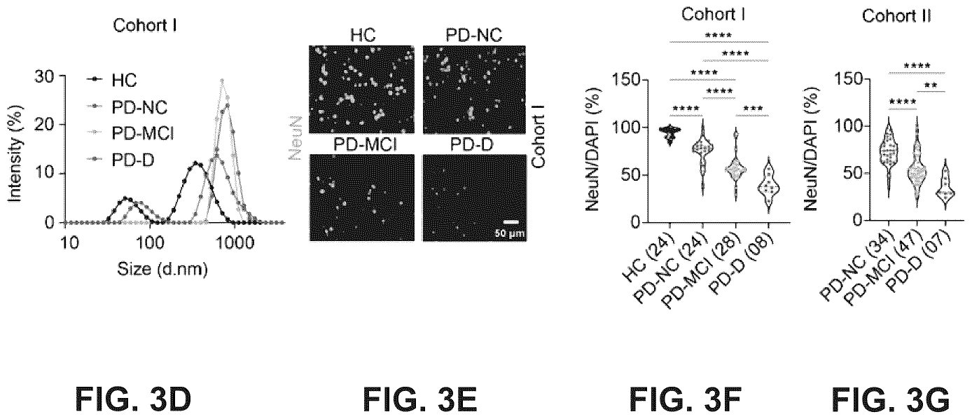

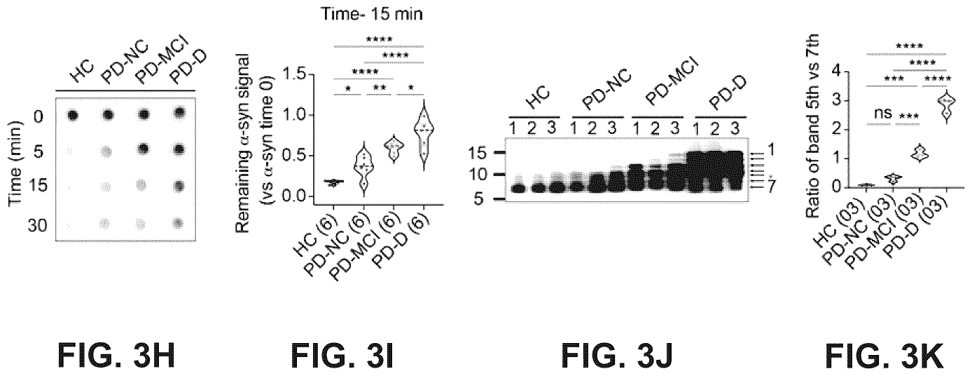

Because both Johns Hopkins University (JHU, cohort I) and University of Washington (UW, cohort II) cohorts have CSF samples of PD patients collected in a cross-sectional manner, we applied SAA to amplify α-syn aggregates from samples from these two cohorts. We performed a series of biophysical and cellular studies to characterize the strain features (FIGS. 2 and 3), which were subsequently analyzed to correlate with clinical features. The thioflavin T (ThT) results showed that both the maximal fluorescence intensity and Lag time (t_{lag}) significantly increase the odds ratio of a PD-CI (including PD-MCI and PD-D) diagnosis over PD-NC when controlling for gender, education, age at baseline visit, baseline MoCA (Montreal Cognitive Assessment) score, total Levodopa dosing, Hamilton Depression score, MDS-UPDRS motor score and disease duration (P=XX) (Table 1).

TABLE 1
Demographic and clinical characteristics of JHU cohort

| | | | PD-Normal | | | | | | |
|---|---|---|---|---|---|---|---|---|---|
| | | | Cognition | | PD-MCI | | PDD | | |
| | PD | Control | (n = 24) | | (n = 28) | | (n = 8) | | p-value |
| Age, years (SD) | | | 60.43 | (7.81) | 66.06 | (6.72) | 71.65 | (6.28) | <0.01 |
| Gender, % male | | | 50 | | 71 | | 100 | | 0.03 |
| Race, % White | | | 100 | | 93 | | 88 | | 0.30 |
| Education, years (SD) | | | 17.83 | (2.39) | 17.71 | (2.02) | 17.38 | (2.77) | 0.89 |
| Levodopa Equivalent | | | 805.03 | (534.60) | 640.15 | (441.59) | 422.75 | (217.84) | 0.12 |
| dosing, mg (SD) | | | | | | | | | |
| MoCA total score (SD) | | | 28.21 | (1.69) | 26.36 | (2.00) | 20.50 | (5.18) | <0.01 |
| MDS-UPDRS part I | | | 10.58 | (4.37) | 10.14 | (4.77) | 11.88 | (7.00) | 0.68 |
| MDS-UPDRS part II | | | 10.17 | (3.78) | 10.89 | (7.24) | 15.63 | (9.20) | 0.11 |
| MDS-UPDRS part III | | | 27 | (8.22) | 30.46 | (10.49) | 44.38 | (11.48) | <0.01 |
| MDS-UPDRS part IV | | | 4 | (3.61) | 3.39 | (3.62) | 1 | (2.83) | 0.12 |
| MDS-UPDRS Total | | | 51.75 | (10.97) | 54.89 | (20.29) | 72.88 | (28.13) | 0.02 |
| Hoehn and Yahr | | | | | | | | | |
| Stage 2 | | | (18) | | (21) | | (3) | | 0.11 |
| Stage 2.5 | | | (3) | | (4) | | (3) | | |
| Stage 3 | | | (3) | | (3) | | (1) | | |
| Stage 4 | | | (0) | | (0) | | (1) | | |
| Hamilton Depression | | | 5.75 | (4.48) | 5 | (3.46) | 7.75 | (5.44) | 0.27 |
| Hamilton Anxiety | | | 6.46 | (4.59) | 7.79 | (3.92) | 8.88 | (2.95) | 0.29 |
| UPSIT | | | 21.96 | (7.10) | 20 | (8.03) | 15.75 | (7.36) | 0.14 |
| RBD symptomatology | | | 66 | | 46 | | 63 | | 0.32 |
| Disease duration | | | 7.10 | (4.19) | 5.27 | (3.59) | 6.03 | (2.98) | 0.23 |

Cross-Sectional: Faster Aggregation and Higher ThT Signal of Amplified α-Syn in PD-D and PD-MCI than PD-NC

The PD patients were divided according to their cognitive diagnoses (PD-NC, PD-MCI, and PD-D), and their CSF samples were amplified with SAA for the ThT assay (FIG. 2a). We found that ThT maximal fluorescence intensity (ThT-mfi) with PD-D is the highest amongst individuals (FIG. 2b) in Cohort I. The ThT-mfi then decreases in a step-wise fashion with significant differences between PD-D and PD-MCI, PD-MCI and PD-NC, and PD-NC and HC (FIG. 2b). The ThT-mfi of the HC group exhibited minimal fluorescence intensity (FIG. 2b). The lag time (ThT t_{lag}), the time at which ThT fluorescent signal appeared from initiating aggregation, was also different between the cognitive groups. The PD-D group spent the least ThT-t_{lag} compared to the PD-MCI and PD-NC groups (FIG. 2c). The PD-MCI group spend less ThT-t_{lag} than the PD-NC group (FIG. 2c). There is no ThT-t_{lag} for the HC group due to the minimal fluorescence intensity (FIG. 2b,c). Moreover, we assessed the half-time (ThT-t₅₀) after sigmoidal fitting to ThT fluorescence spectra, and found that ThT-t₅₀ of PD-D was significantly lower than ThT-t₅₀ of the PD-MCI and PD-NC groups (FIG. 2d). These ThT results, including ThT-mfi, ThT-t_{lag}, and ThT-t₅₀ were replicated in Cohort II (FIG. 2e-g), showing a similar pattern of ThT spectra in Cohort I.

### Cross-Sectional: Homogenous Size Distribution of Amplified α-Syn Aggregates in PD-D and PD-MCI.

Dynamic light scattering (DLS) is a laser scattering technique that characterizes the size distribution of diffusing nano- and micro-particles. We applied DLS to assess the size profiles (i.e. peak number, intensity, size) of amplified α-syn aggregates (FIG. 3a) stratified by cognitive strata. The typical DLS spectra of both PD-MCI and PD-D groups showed significantly greater homogeneity (one peak) compared to the PD-NC and HC groups (two peaks) (FIG. 3b,c). This difference in peak number between PD-NC and PD-CI was identified in Cohort I (FIG. 3b) and replicated in Cohort II (FIG. 3c).

The typical DLS spectra of α-syn strains are shown in FIG. 3d. Because the DLS machine automatically recognizes the peak with high intensity as peak 1 (major), and the peak with low intensity as peak 2 (minor). While the HC and PD-NC groups have the same peak number (two), PD-MCI and PD-D groups have the same peak number (one), differences in the peak size and peak intensity did distinguish between these groups in Cohort I and in Cohort II.

The morphological analysis of amplified α-syn aggregates by transmission electron microscopy (TEM) shows difference in the fibrils length. The amplified α-syn aggregates from the PD-NC group shows mixture of long and short fibrils. In contrast, amplified α-syn aggregates from the PD-MCI and PD-D groups shows similar fibril length. These results are consistent with DLS results of homogenous size distribution in PD-MCI and PD-D.

Combined with the number and the distribution of peaks, the SAA-amplified products of HC, PD-NC, and PD-MCI/PD-D are distinguishable. Peak number continued to predict PD-NC from PD-CI when controlling for age, education, and disease duration and when either controlling for case status or when analyzing the PD participants only (FIG. 2). These differences were identified in both Cohort I and Cohort II (FIG. 2). As we would expect based on the mean values, the peak number did not predict conversion from PD-MCI to PD-D (FIG. 2). DLS peak number did distinguish between PD-NC and PD-CI participants with reasonable sensitivity and specificity but did not adequately distinguish between PD and HC participants (FIG. 2).

Cross-Sectional: Amplified α-Syn Aggregates of PD-D Induce More Neurotoxicity than PD-MCI and PD-NC

To determine the pathological role, we administered these amplified α-syn aggregates into the mouse primary cortical neurons and assessed neurotoxicity with NeuN (neuronal nuclear marker) immunostaining (FIG. 3e). In Cohort I, amplified α-syn aggregates from the PD-NC group exhibited more neurotoxicity than the HC group (FIG. 3e,f). The PD-MCI group significantly showed increased neurotoxicity than PD-NC, and the PD-D group exhibited the highest neurotoxicity (FIG. 3e,f). The neurotoxicity results of Cohort II similarly showed that the PD-D group exhibited the highest neurotoxicity (FIG. 3g), and PD-MCI showed a significant enhancement of neurotoxicity than the PD-NC group (FIG. 3g).

As with the ThT and DLS data, neurotoxicity as measured by NeuN immunostaining predicted cognitive status (FIG. 3) and identified individuals with PD compared to healthy controls as well as PD-NC compared to PD-CI (FIG. 3).

### Cross-Sectional: Amplified α-Syn Aggregates in the PD-NC, PD-MCI, and PD-D Groups are Different Strains

We performed proteinase K (PK) digestion on these amplified α-syn aggregates over time, and followed with the dot-blot assay (FIG. 3h) and silver staining assays (FIG. 3j), to determine strain distinction. In the dot-blot assay, the PD-D strain showed the strongest resistance to PK digestion as evidenced by the remaining α-syn signal (FIG. 3h,i); the PD-MCI strain exhibited less resistance than PD-D strain, but more resistance than PD-NC strain (FIG. 3h,i); the PD-NC strain exhibited mild resistance to PK digestion; the HC strain has minimal resistance to PK digestion (FIG. 3h,i). Furthermore, we performed the silver staining and assessed the digested band patterns in response to PK digestion (FIG. 3j). Because these strains have diverse resistance to PK digestion, we chose to compare the digested bands between the 5^{th} and 7^{th} bands. The results showed significant differences between the PD-D and PD-MCI strains (FIG. 3j,k), PD-MCI and, PD-NC strains. However, there was no difference between the PD-NC and HC strains (FIG. 3j,k).

### Cross-Sectional: Artificial Intelligence (AI) Analysis of Combined 3 and 9 Features of Amplified α-Syn Strains in PD-NC, PD-MCI, and PD-D

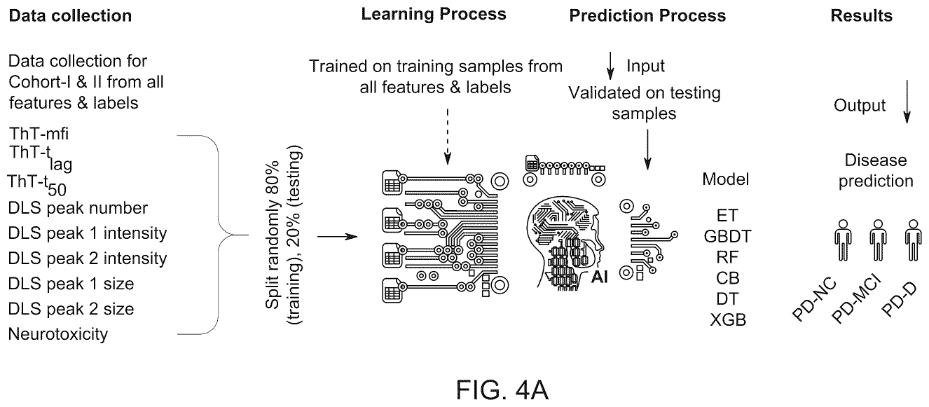

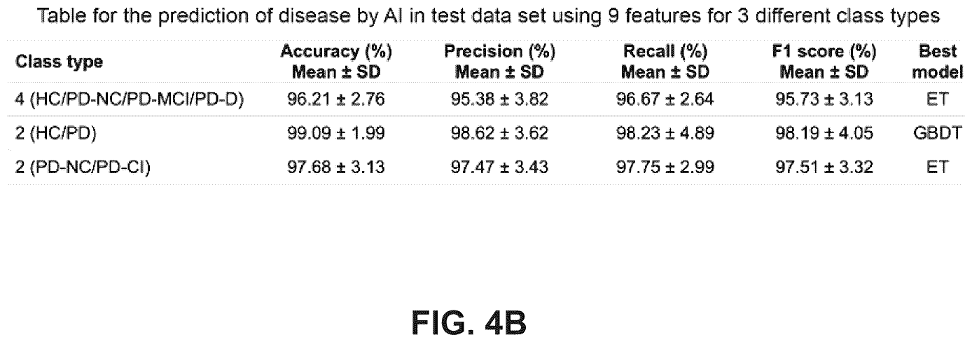

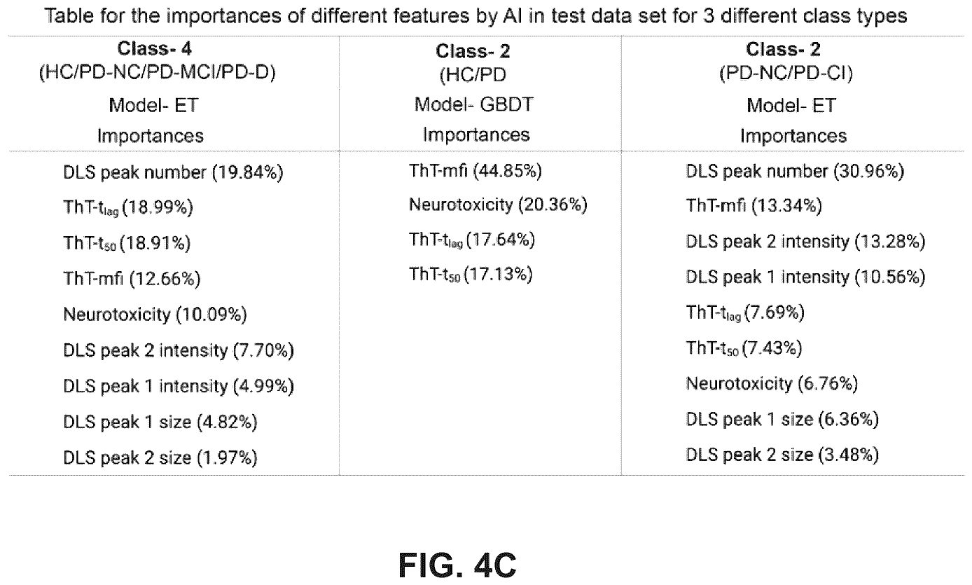

For our AI analysis, we conducted three types of classification tasks. These tasks included the 4-class classification of the discrimination of HC/PD-NC/PD-MCI/PD-D, as well as the binary classification tasks of HC vs. PD (PD-NC, PD-MCI, PD-D (Appendix A)), PD-NC vs. PD-CI (PD-MCI, PD-D). Six machine learning models were employed: Decision Tree (DT), Gradient Boosted Decision Tree (GBDT), Random Forest (RF), Extreme Gradient Boosting (XGB), Cat Boost (CB), and Extra Trees (ET) (FIG. 4a). To overcome the limitations posed by the small dataset sizes in Cohort I and II, we propose a comprehensive strategy. This involves merging the two cohorts and randomly splitting the combined dataset into training-testing sets, maintaining an 80%-20% ratio (FIG. 4a). To further ensure robust model performance, we employ cross-validation for training and validating the models. By adopting this approach, we aim to address the challenges associated with insufficient training samples, potential data skew, and class imbalance, thereby significantly enhancing the reliability and effectiveness of the model's output.

Single features of the ThT profile. Initially, we trained and validated the three classification tasks individually using the three ThT features (ThT-mfi, ThT-t_{lag}, and ThT-t₅₀). The results indicated that for the 4-class classification, the DT model utilizing the ThT-t_{lag} feature achieved the highest mean accuracy of roughly 88.03%. When distinguishing between PD and HC in the 2-class classification, all models demonstrated exceptional performance using either one of the 3 ThT features, with almost all models achieving a high mean accuracy of 95.46%. For the 2-class classification (PD-NC vs. PD-CI), the GDBT model using the ThT-t_{lag} feature achieved the highest mean accuracy of about 90.89%. In general, it was observed that the ThT-t_{lag} feature often resulted in better model performance, suggesting it might be a more critical feature for PD classification. Moreover, the two 2-class classification tasks appeared to be easier for the AI to learn than the 4-class classification task.

Combined 3 features of ThT profile. After, that we tried to combine the ThT 3 features all together to train and validate on the very same models. The results were as follows: The ET model outperformed others in the 4-class classification, achieving the highest mean accuracy of 88.94% and an F1 score of 90.28%. The ET model continued to excel in the 2-class classification (HC vs. PD), securing the highest mean accuracy of 95.61% and an F1 score of 92.64%. In contrast, the XGBoost model delivered superior results in the 2-class classification (PD-NC vs. PD-CI) with the highest mean accuracy of 91.43% and an F1 score of 90.59%. Subsequently, we selected the top-performing models from the single feature of the ThT 3 features and conducted a students t-test against the combined ThT 3 features models. It was observed that in all three types of classification tasks, the models trained on the combined ThT 3 features slightly outperformed those trained on a single best model. However, the statistical analysis using a students t-test revealed that the observed differences had P values ranging from 0.5 to 0.87, suggesting that, according to conventional criteria, these differences are not considered statistically significant.

Single features of DLS and neurotoxicity. The DLS data including peak number, peak size, and peak intensity followed a similar result pattern showing that DLS results can predict cognitive impairment in two class types of PD-NC vs. PD-CI but not in four class types. The DLS data applied to our AI analysis was not able to predict two class types HC vs PD (FIG. 3d). When using the neurotoxicity data the AI analysis was able to predict all 3 kinds of classification tasks in some level but quite struggling.

Combined 9 features. Given the characteristics of the above features for the three classification tasks, we hypothesized that their integration could potentially improve prediction accuracy. We assembled a comprehensive set of nine features for AI analysis, which included ThT (ThT-mfi, ThT-t_{lag}, ThT-t₅₀), DLS (peak number, size of peak ½, intensity of peak ½), and neurotoxicity. After training and evaluating the same models, we found that: In terms of 4-class classification, the ET model outperformed others, achieving the highest mean accuracy (96.21%) and F1 score (95.73%) (FIG. 4b). For the 2-class classification (HC vs. PD), the GBDT model was superior, with the highest mean accuracy (99.09%) and F1 score (98.19%). For the 2-class classification (PD-NC vs. PD-CI), the ET model was the most effective, with the highest mean accuracy (97.68%) and F1 score (97.51%) (FIG. 4b). The standard deviations of these metrics suggest a generally consistent performance across the models. In addition to predictive metrics, we also utilized tree models to determine the significance of various features. Upon examining the data results for feature importance, we discovered that the feature ‘ThT-t_{lag}’ consistently held high importance across all models and classes, frequently ranking as the most or second most important feature. Similarly, the feature ‘ThT-mfi’ and ‘ThT-t₅₀’ consistently ranked among the top three most important features across all models and classes. These findings suggest that ‘ThT-t_{lag}’, ‘ThT-mfi’, and ‘ThT-t₅₀’ are potent predictors of all types of classification tasks mentioned above.

In conclusion, using nine features combined appears to be more effective and consistent in classifying PD than using any single feature. However, ‘ThT-t_{lag}’, ‘ThT-mfi’ and ‘ThT-t₅₀’ emerge as the most significant features in this context.

Longitudinal: ThT Profile Changes of Amplified α-Syn Strains from the Same Individuals when Cognition Progressed from PD-NC to PD-MCI and PD-D

Longitudinal: ThT-mfi: Because the cross-sectional results showed that PD-NC and PD-CI patients have distinct α-syn strains, we hypothesized that the strains change from PD-NC to PD-CI in the same individuals. To evaluate this question, we utilized the longitudinal clinical follow-up, consensus conference cognitive diagnosis, and available longitudinal CSF collection from Cohort I. The CSF samples from the same individuals in the initial and last visits (either a 3-, 4-, or 5-year follow-up time) of Cohort I were used to amplify α-syn strains followed by characterization and correlation studies. The results showed no significant changes in ThT-mfi if the cognitive status had no change between the initial and last visits. That is, patients who remained controls, PD-NC, PD-MCI, or PD-D had a stable ThT-mfi that matched their cognitive strata. Strikingly, the ThT-mfi significantly increased in the individuals whose cognition declined in the last visit compared to the initial visit: PD-NC→PD-MCI, and PD-MCI→PD-D. Because the ThT results are correlated with the longitudinal cognitive decline, we further increased the time resolution to determine whether the ThT profile can predict cognitive decline. In the ThT studies of α-syn strains amplified from these yearly collected longitudinal CSF samples, the results showed the ThT-mfi remained the same in those individuals without cognitive change, consistent with the ThT results of the first-last visit. In those individuals, PD-NC→PD-MCI, and PD-MCI→PD-D, the results showed that the ThT-mfi increased when cognition progressed from PD-NC to PD-MCI, and PD-MCI to PD-D.

Longitudinal: ThT-t_{lag} and ThT-t₅₀: Similarly, ThT-t_{lag} and ThT-t₅₀ were significantly reduced in individuals whose cognition changed: PD-NC→PD-MCI, and PD-MCI→PD-D. The groups without cognitive change PD-NC→PD-NC, and PD-D→PD-D did not show any change in ThT-t_{lag} or ThT-t₅₀. These two ThT features change when cognitive decline. In brief, the changed ThT-mfi, ThT-t_{lag}, and ThT-t₅₀ occurred only at the same time as the progression to PD-MCI or PD-D, therefore not predating the cognitive decline.

Longitudinal: The Amplified α-Syn Strains at the PD-CI Stages Become More Homogenous than PD-NC; the Change of DLS Peak Number Occurs at the PD-NC Stage One Year Before the Diagnosis of PD-MCI

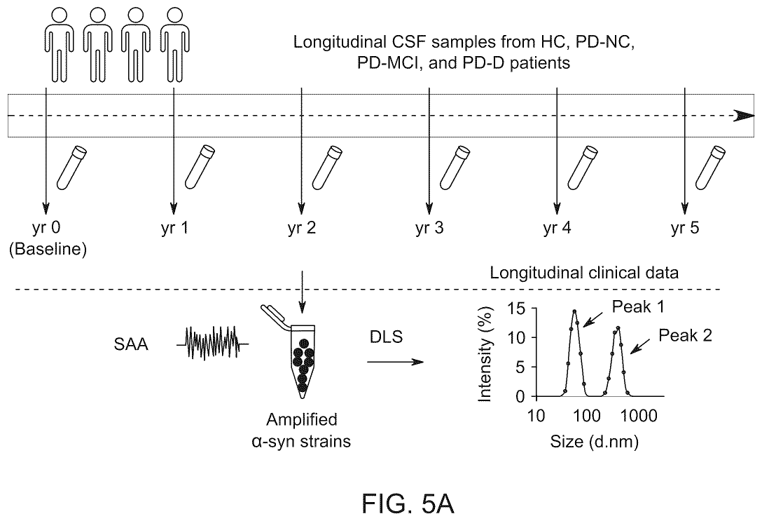

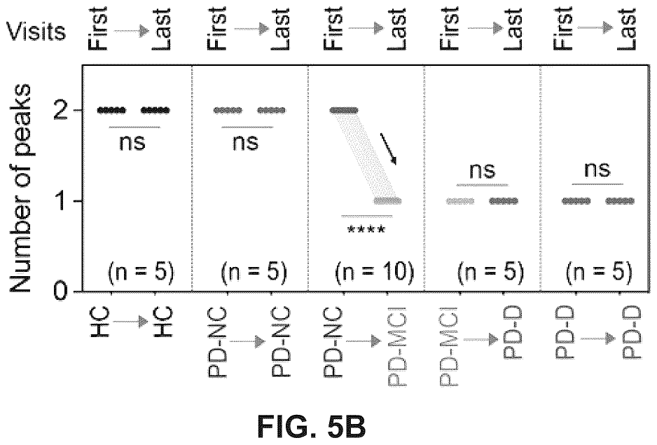

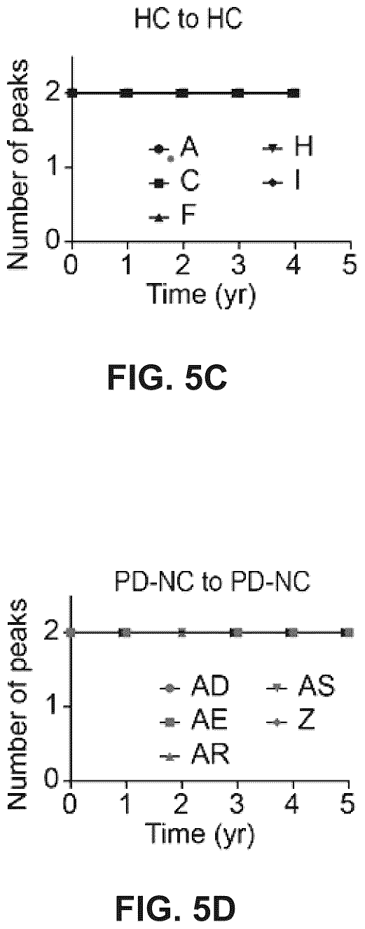

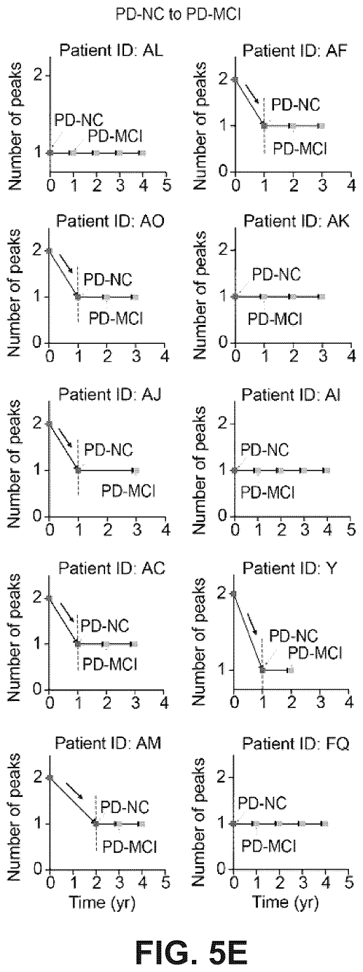

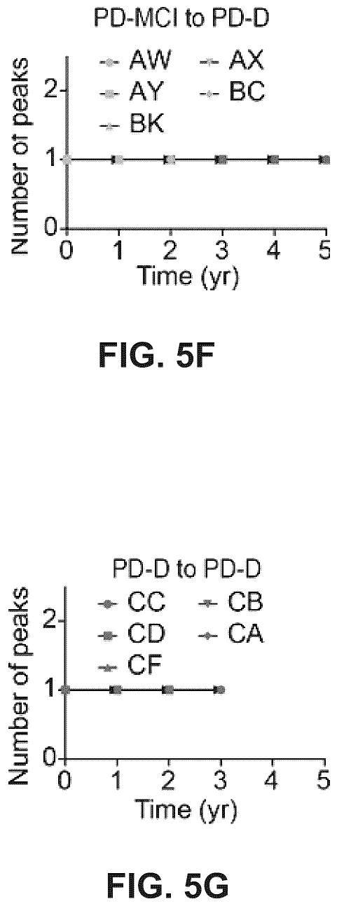

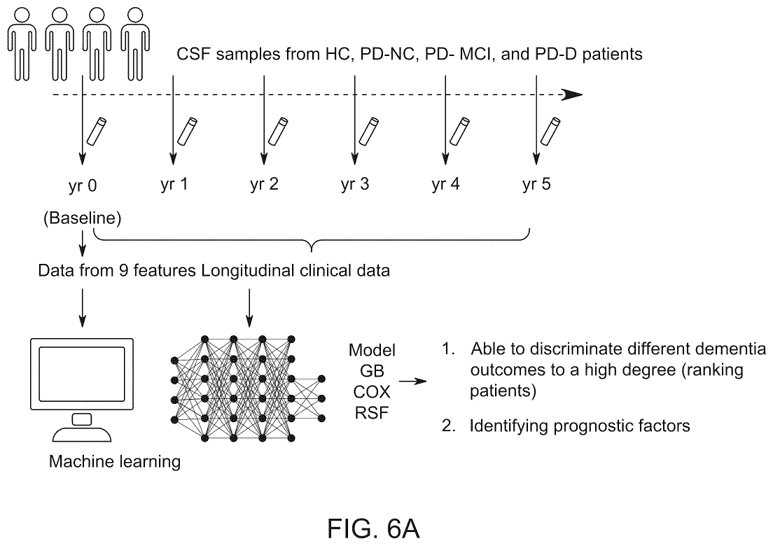

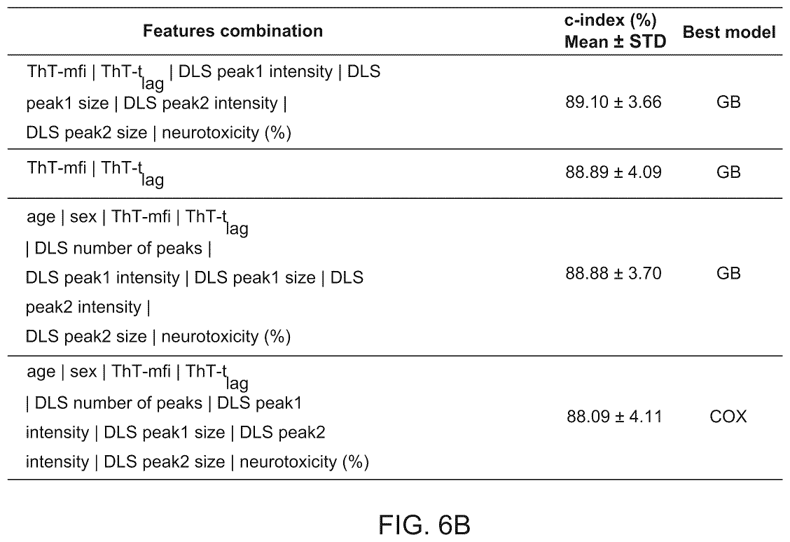

We then sought to determine the correlation between the DLS results and longitudinal cognitive status (FIG. 5a). In the DLS assay to compare the first and the last visits, only the group of PD-NC→PD-MCI showed the peak number change (from 2 to 1) (FIG. 5b). The peak number of the other groups has no change, including HC→HC, PD-NC→PD-NC, PD-MCI→PD-D, and PD-D→PD-D (FIG. 5b). In the yearly mapping, the peak number remained the same in those individuals without cognitive change (FIG. 5c,d,f,g). In Cohort I, there is a total of 10 patients developed from PD-NC to PD-MCI. Strikingly, we observed the peak number of amplified α-syn from all these 10 patients at the PD-NC stage went down to 1 at the visit one year before the diagnosis of PD-MCI (FIG. 5e). In brief, the longitudinal DLS results are consistent with the cross-sectional results that, the peak number only changes when PD-NC develops to PD-MCI. Of note, the peak number changes at the PD-NC stage one year before the diagnosis of PD-MCI. The peak number will not change with disease duration or development to PD-D.

Longitudinal: Amplified α-Syn Strains Exhibited More Neurotoxicity that Depended on Cognitive Decline, but not Disease Duration

We then assessed the longitudinal samples to determine whether there was increased neurotoxicity that correlated with cognitive decline and/or disease duration. The amplified α-syn strains were administered into the mouse primary cortical neurons for neurotoxicity evaluation. In the comparison between the first and the last visits, a significant increase in neurotoxicity was observed in the groups with cognitive decline, specifically the PD-NC→PD-MCI, and PD-MCI→PD-D groups. In those groups without cognitive change during the follow-up time (HC→HC, PD-NC→PD-NC, and PD-D→PD-D), there were no significant changes in neurotoxicity. We further assessed the neurotoxicity of these α-syn strains amplified from yearly-collected CSF. In the groups of HC→HC, PD-NC→PD-NC, and PD-D→PD-D, the results showed that the neurotoxicity remained the same yearly. During the progression from the PD-NC to PD-MCI, and PD-MCI to PD-D, the enhanced neurotoxicity was correlated with the onset time of diagnosed cognitive impairment. In brief, the neurotoxicity of amplified α-syn strains increases when PD patients have cognitive decline, but remains the same when the cognitive status is stable. The neurotoxicity increase occurred only at the same time as the progression of PD-NC to PD-MCI, or PD-MCI to PD-D, therefore not predating the cognitive decline.

Longitudinal: The Amplified α-Syn Strains from the Same Individual Show More Resistance to PK Digestion when Cognition Progressed from PD-NC to PD-MCI and PD-D

To further evaluate the longitudinal strain changes, we performed the dot-blot assay and assessed the remaining α-syn signal after PK digestion. Within the groups without cognitive change, HC→HC, PD-NC→PD-NC, and PD-D→PD-D, there was no significant difference in the remaining α-syn signal between the first and the last visits. In the PD-NC→PD-MCI group, the amplified α-syn strains at the PD-MCI stage were significantly more resistant to PK digestion than PD-NC strains in the same individuals. In the PD-MCI→PD-D group, PD-D strains were significantly more resistant than PD-MCI strains in the same individuals. We further mapped the yearly strain change. The results showed stable strain from patients without cognitive decline and more resistance to PK digestion from patients with cognitive change: PD-NC→PD-MCI, PD-MCI→PD-D. In brief, increasing resistance to PK digestion is dependent on cognitive impairment, but not on the disease duration.

Longitudinal: Survival Analysis Results Reveal that the ‘ThT-Mfi’ Feature and the Combination of Multiple Features Notably Impact the Prediction Models

In our cross-sectional AI study, we observed that the features of α-syn strains provide valuable information for distinguishing between PD-CI and PD-NC. This led us to question whether these features could also predict the timeframes and likelihoods for patients currently classified as PD-NC to transition into PD-CI status. To explore this, we employed survival analysis models, utilizing a variety of feature sets and hyperparameters. The three models we used were the Cox proportional hazards model (Cox), gradient boosting survival analysis (GB), and random survival forest (RSF). We experimented with different combinations of features to evaluate their effectiveness. We tested three ThT features of α-syn strains, one feature of neurotoxicity, and two demographic features (age and sex) individually against the three survival analysis models. Subsequently, we tested various combinations of ThT features, followed by all α-syn strain features, and finally, a combination of all α-syn strain features with age and sex.

As a result in the single feature situation, the ‘ThT-mfi’ consistently shows high c-index mean values across all models (Cox, GB, and RSF), indicating that it is a strong predictor. The combination of features ‘ThT-mfi’, ‘ThT-t_{lag}’, ‘DLS-peak1-intensity’, ‘DLS-peak1-size’, ‘DLS-peak2-intensity’, ‘DLS-peak2-size’, ‘neurotoxicity’ also shows high c-index mean values. This suggests that these features together can provide a good prediction. The Time-Dependent ROC (receiver operating characteristic) AUC (area under the curve) mean values are generally lower, indicating that the models may not be as effective at distinguishing between those who experience the event and those who do not.

### DISCUSSION

A major finding of this example is the identification of the correlation between α-syn strain changes and cognitive decline in PD. We took advantage of longitudinal follow-up of PD patients and CSF sample collection and amplified α-syn aggregates. Both cross-sectional and longitudinal studies showed a strong correlation between α-syn strain features and cognitive status. α-Syn strains exhibited differences in the three cognitive stages (PD-NC, PD-MCI, and PD-D). Multiple features of α-syn strains with AI analysis can better predict the diagnosis of PD-NC, PD-MCI, and PD-D. Longitudinal features of α-syn strains can predict cognitive decline. Of note, α-syn strain changes at the PD-NC stage one year before the diagnosis of PD-MCI.

The tool development for the characterization of α-syn strain is important. The breakthrough of cutting-edge technologies in biophysics, biochemistry, and cellular assays combined with AI analysis, will significantly facilitate the development of strain biomarker that is correlated with clinical phenotypes. Other seed templates can be considered, such as blood, urine, saliva, etc., which can provide more sample frequency for the longitudinal correlation study. Also, more work is needed to optimize the amplification and characterization methods, which can better correlate with clinical data. The association between the molecular signature of prion-like seeds and disease progression is also urgently needed. These technologies can be further applied to discriminate diverse proteinopathies, such as α-synucleinopathies and tauopathies.

It is very compelling that these amplified α-syn from CSF can be used as biomarkers for cognitive status in PD. However, the unanswered questions are: whether these α-syn strains exist in the PD-NC, PD-MCI, and PD-D brains, which drives the disease progression, e.g., cognitive impairment? What causes the α-syn dynamics? If these PD-MCI and PD-D strains can be identified as the driver for the cognitive impairment, it provides the rationale to develop antibodies/nanobodies⁸ and inhibitors to these specific strains.

### Methods

### Patient Enrollment and Biosample Collection

### Johns Hopkins University (Cohort I)

Individuals with PD and those without motoric evidence of parkinsonism (healthy controls) were enrolled at JHU as part of our participation in the NINDS Parkinson's Disease Biomarker Program (PDBP). Those with PD had to meet UK Brain Bank criteria for idiopathic PD, modified to allow for individuals with a family history of PD, and be taking levodopa therapy for their PD. The healthy controls needed to have a normal Montreal Cognitive Assessment score (MoCA>25) and not have a first-degree relative with parkinsonism. All participants had to agree to and be eligible for an annual lumbar puncture. All individuals underwent the PDBP standard set of motor, psychiatric, and cognitive assessments every 6 months for the first 5 years of the investigation as well as a one-time 12-month visit at the end of the investigation, and an annual lumbar puncture.

### Clinical and Cognitive Assessments and Diagnosis Determination

Individuals in both Cohort I and Cohort II also underwent additional annual neuropsychiatric testing consisting of two tests per cognitive domain as well as a structured interview of both the participant and an informant to determine the clinical dementia rating scale. Research staff from both cohorts (LST, CB, CZ, BC) then met monthly to review the clinical and cognitive diagnosis as part of the consensus conference diagnosis process for Cohort I. The same process occurred for Cohort II with a different set of research staff (CZ, BC, XX, XX). Individuals were divided into HC and PD based on motoric symptoms and then divided into normal cognition, mild cognitive impairment (cognitive impairment no dementia (CIND)), and dementia according to published diagnostic criteria.

### Expression and Purification of α-Synuclein Protein

Recombinant human α-syn protein were prepared according to the previous method. pRK172-α-syn plasmid was transduced in BL21 (DE3) cells and cultured at 37° C. in lysogeny broth overnight. The E. coli pellets were resuspended with osmotic shock buffer (3.63 g Tris-base, 400 g sucrose, and 0.744 g EDTA were dissolved in 1 L DDW, pH 7.2) by drastic agitation. The mixture was then centrifuged at 10,000 g for 30 min to remove the supernatant, and DDW containing proteinase inhibitor and 80 μL saturated MgCl₂ were added to resuspend the pallets. The supernatant was collected (10,000 g, 30 min centrifugation) and filtered through a 0.45 μm filter, followed by the dialysis with low salt buffer (20 mM Tris-base, 50 mM NaCl in DDW; pH 8.0) overnight at 4° C. α-Syn protein was purified with fast protein liquid chromatography (FPLC) and saved in a −80° C. freezer. The purity was evaluated with Coomassie brilliant blue staining and immunoblot. The concentration was measured with a BCA assay.

Amplification of Pathogenic α-Syn in Patient CSF with SAA

The amplification of α-syn strains from patient-derived CSF samples was performed using the SAA method by referring to previous work with some modifications. The SAA equipment containing the microplate horn (#431MPX), a sound enclosure (#432MP), and a thermoelectric chiller (#4900) was purchased from Qsonica. Briefly, recombinant α-syn was centrifuged at 100,000 g for 30 min at 4° C. to remove any preformed aggregates before use. Then, α-syn was diluted with SAA buffer (1% Triton X-100 in PBS), and transferred 100 μL into PCR tubes containing a suitable amount of silicon beads (diameter 1.0 mm, purchased from BioSpec products), and 10 μL CSF samples were added as seeds in triplicate. The final concentration of α-syn was 0.3 mg/mL. After mixing, the samples were subjected to sonication (Amplitude: 5; 40 sec sonication and 29 min 20 sec incubated at 37° C.). In total 40 amplification circles for 1-day reaction and 280 amplification circles for 7-day reaction. 5 μL samples were collected every day and amplification was monitored by measuring Thioflavin T (ThT) (Sigma-Aldrich, cat No. T3516) fluorescence using a Fluorescence Spectrophotometer (Varioskan LUX plate reader, Thermo Fisher Scientific) with fixed excitation and emission wavelength at 450 nm and 485 nm respectively. After 7 days, the mixture was transferred into the centrifugal filter (Millipore, MCW: 3000) containing 15 mL PBS, and centrifuged at 4000 g for 30 min at 4° C. to remove Triton X-100. The washing with 15 mL PBS and centrifugation was repeated 8 times and the final SAA products were collected. SAA products were spun for 30 min at 20,000 g, the amount of monomeric α-syn in the supernatant was assessed by BCA assay and the pelleted assemblies were resuspended in PBS buffer.

### ThT Fluorescence Assay

The SAA sample (5 μL) was taken out and added into a 55 μL ThT solution (20 μM). Samples were subsequently plated in triplicate on 384 well black/clear bottom plates (Sigma-Aldrich, cat no. P6491), and the fluorescence was measured at 450/485 nm excitation/emission with a microplate reader (Varioskan LUX plate reader, Thermo Fisher Scientific). Following data acquisition, the kinetic curves were fitted to Equation. 1 to calculate the half-time (t₅₀) value and lag time (t_{lag}) for each curve.

$$y = A 2 + ( A 1 - A 2 ) / ( 1 + exp ( ( x - {x}_{0} ) / dx ) ) \qquad Equation . 1$$

where A1 represents initial fluorescence, A2 is the final fluorescence value, x₀ is the half-time (t₅₀) value, and dx represents the time constant.

$${t}_{lag} = {x}_{0} - 2 dx . \qquad Equation . 2$$

### Proteinase K Digestion, Dot Blot, and Silver Staining

The SAA samples (7 μg) were mixed with proteinase K (PK) and incubated at 37° C. at different time points (0, 5, 15, and 30 min). For the dot blot assay, the PK-digested SAA samples were loaded onto the nitrocellulose membrane (Bio-Rad, cat no. 1620112) and blocked by 5% bovine serum albumin (BSA) (Sigma-Aldrich, cat no. A7906) in TBST for 1 hr at room temperature (RT). The membrane was then transferred into the mouse anti-α-syn mAb (1:2000 dilution, BD Biosciences, cat no. 610787) in TBST with 5% BSA overnight at 4° C. Following with TBST wash (3 times×5 min), the membrane was incubated with anti-mouse IgG-HRP (1:5000 dilution, GE Healthcare, cat no. NA931) for 1 hr at RT. After TBST wash, the signal was developed with SuperSignal West Pico Plus chemiluminescent substrate (Thermo Fisher Scientific, cat no. 34096). For the silver staining, PK-digested SAA samples were loaded on the SDS-PAGE (15%) gels. Silver staining was performed using Pierce Silver Stain Kit (Thermo Fisher Scientific, cat no. 24612). All the images were acquired and processed with Amersham Image 600 (GE Healthcare Life Sciences).

### Dynamic Light Scattering (DLS)

The SAA sample (10 μg) was mixed with filtered phosphate buffer (990 μL). Measurements were performed using a Zetasizer Nano-ZS (Malvern Instruments, Malvern, UK) equipped with a He—Ne laser. Each sample was measured in 1-cm path-length polystyrene semi-micro disposable cuvettes (Fisher Emergo, Landsmeer, The Netherlands). The cell holder was maintained at 25° C. For each sample, 10 runs were performed, with three repetitions.

### Transmission Electron Microscopy (TEM)

The SAA sample (7 μL) was mounted on 400 mesh carbon-coated copper grids (Electron Microscopy Sciences, cat no. CF400-CU-50) Sample was incubated on a grid for 30 sec at room temperature and then washed by double-distilled (dd)-water for 30 sec. Excess liquid was removed using lint-free tissue paper. Further, samples were negatively stained with 2% uranyl acetate (Electron Microscopy Sciences, cat no. 22400) for 1 min. The grids were air-dried overnight, and images were recorded by TEM (Hitachi H7600 TEM, Tokyo, Japan) with accelerating voltage at 80 kV. The time of sample preparation and concentration of sample for TEM were identical for all samples. The length of the fibrils was measured using ImageJ.

### Primary Cortical Neuron Culture, Neurotoxicity of SAA Products

Mouse primary cortical neurons were cultured from embryonic 15.5-day pups of CD-1 pregnant mice (Charles River). 48 well plates were coated via Poly-L-ornithine solution (0.2 mg/mL) for 1 hr at 37° C. and washed 3 times with sterile dd-water. Primary neurons at 7 days in vitro (DIV) were treated with SAA samples with a final concentration of 10 μg/well. The neuropathology and the neurotoxicity were assessed at 21 and 28 DIV individually. The primary cortical neurons were washed with PBS, fixed in 4% paraformaldehyde (PFA), followed by blocking in 3% goat serum containing PBST (0.1% Tween-20) for 1 hr. Anti-NeuN (1:250, MAB377, Sigma-Aldrich) were incubated overnight at 4° C., followed by Alexa-fluor 488 secondary antibodies (1:2000, Thermo Fisher Scientific) and Hoechst (1:5000, Thermo Fisher Scientific) at RT for 1 hr. The fluorescence images were obtained via a Nice microscope (Zeiss). The number of NeuN was quantified using ImageJ software (National Institute of Health, Bethesda, MD).

### Statistical Analysis

Statistical analysis were performed using the statistical software Stata (version 18). The baseline demographic and clinical characteristics of the participants are presented as mean+/−standard deviation (SD) or number (%). The characteristics were compared using the student's t-test or chi squared as appropriate. Logistic regression analyses were performed with the binary cognition variable as the outcome variable and the biomarker of interest as the independent variables, adjusting for covariates, such as age and gender. Receiver operating characteristic curves were calculated to visualize and compare the predictivity of the biomarkers. The Cox proportional hazard model was used to determine whether baseline biomarker data is associated with the progression to cognitive impairment. Two-sided p-values<0.05 were considered significant.

### Disease Prediction Using Machine Learning/Artificial Intelligence (AI)

Cross-sectional classification: We implemented machine learning classifiers to solve the cross-sectional classification problem using patient sample data. The data, consisting of 9 α-syn strain features and classification labels, was preprocessed and converted into numerical form. We selected six tree-based models and used grid search to find the optimal hyperparameters for each model. The models were trained and evaluated using cross-validation with 20 iterations. Evaluation metrics such as accuracy, precision, recall, and F1 score were calculated, and feature importance were analyzed. By considering the mean and standard deviation of the evaluation metrics, we determined the best model and hyperparameters for each classification problem. More details can be found in Supplementary Methods.

Survival Analysis: We utilized longitudinal data for training and evaluating survival analysis models. The process involved loading and preprocessing the data, selecting appropriate models for survival analysis (including Cox Proportional Hazards, Gradient Boosting, and Random Survival Forest), conducting optimal hyper-parameter search, training the models, and calculating performance metrics. The chosen metrics were the Concordance Index (C-index), which measures predictive accuracy, and the Time-Dependent ROC AUC, which evaluates the discriminatory power of the models over time. More details can be found in Supplementary Methods.

Some further aspects are defined in the following clauses:

Clause 1: A method of determining a cognitive status of a test subject, the method comprising determining three or more features from a sample obtained from the test subject using at least a thioflavin T (ThT) assay, a dynamic light scattering (DLS) assay, and a neurotoxicity assay to produce a test subject data set; and using the test subject data set to classify the test subject as having a Parkinson's disease (PD) status of PD-NC (normal cognition) or PD-CI (cognitive impairment), thereby determining the cognitive status of the test subject.

Clause 2: The method of Clause 1, comprising determining four or more, five or more, six or more, seven or more, eight or more, nine or more, ten or more, or more features from the sample.

Clause 3: The method of Clause 1 or Clause 2, wherein the features determined from the ThT assay comprise one or more of a ThT-mfi value, a ThT-t_{lag} value, or a ThT-t₅₀ value.

Clause 4: The method of any one of the preceding Clauses 1-3, wherein the features determined from the DLS assay comprise one or more of a peak number value, a size of peak ½ value, an intensity of peak ½ value.

Clause 5: The method of any one of the preceding Clauses 1-4, wherein when the cognitive status of the test subject is classified as PD-CI, the method further comprises classify the test subject as having PD-MCI (mild cognitive impairment) or PD-D (dementia).

Clause 6: The method of any one of the preceding Clauses 1-5, further comprising repeating the method one or more times using samples obtained from the test subject at subsequent time points to evaluate the cognitive status of the test subject over a selected period of time.

Clause 7: The method of any one of the preceding Clauses 1-6, further comprising predicting a probable disease progression outcome for the test subject based at least in part on the cognitive status of the test subject.

Clause 8: The method of any one of the preceding Clauses 1-7, further comprising administering or discontinuing administering a therapy to the test subject based at least in part on the cognitive status of the test subject.

Clause 9: The method of any one of the preceding Clauses 1-8, further comprising generating a therapy recommendation for the test subject based at least in part on the cognitive status of the test subject.

Clause 10: A computer-implemented method of determining a cognitive status of a test subject, the method comprising: passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and outputting from the model a classification of the test subject as having a Parkinson's disease (PD) status of PD-NC (normal cognition) or PD-CI (cognitive impairment), thereby predicting the determining the cognitive status of the test subject.

Clause 11: The computer-implemented method of Clause 10, wherein the model was created using the reference subject data sets produced by determining four or more, five or more, six or more, seven or more, eight or more, nine or more, ten or more, or more features from samples obtained from the reference subjects.

Clause 12: The computer-implemented method of Clause 10 or Clause 11, wherein the features determined from the ThT assay comprise one or more of a ThT-mfi value, a ThT-t_{lag} value, or a ThT-t₅₀ value.

Clause 13: The computer-implemented method of any one of the preceding Clauses 10-12, wherein when the cognitive status of the test subject is classified as PD-CI, the model further outputs a classification of the test subject as having PD-MCI (mild cognitive impairment) or PD-D (dementia).

Clause 14: The computer-implemented method of any one of the preceding Clauses 10-13, further comprising outputting from the model a prediction of a probable disease progression outcome for the test subject based at least in part on the cognitive status of the test subject.

Clause 15: The computer-implemented method of any one of the preceding Clauses 10-14, further comprising outputting from the model a therapy recommendation for the test subject based at least in part on the cognitive status of the test subject.

Clause 16: The computer-implemented method of any one of the preceding Clauses 10-15, wherein a trained electronic neural network comprises the model.

Clause 17: The computer-implemented method of any one of the preceding Clauses 10-16, comprising determining three or more features from a sample obtained from the test subject using at least a thioflavin T (ThT) assay, a dynamic light scattering (DLS) assay, and a neurotoxicity assay to produce the test subject data set.

Clause 18: The computer-implemented method of any one of the preceding Clauses 10-17, comprising determining four or more, five or more, six or more, seven or more, eight or more, nine or more, ten or more, or more features from the sample obtained from the test subject.

Clause 19: The computer-implemented method of any one of the preceding Clauses 10-18, wherein the features determined from the ThT assay comprise one or more of a ThT-mfi value, a ThT-t_{lag} value, or a ThT-t₅₀ value.

Clause 20: The computer-implemented method of any one of the preceding Clauses 10-19, wherein the features determined from the DLS assay comprise one or more of a peak number value, a size of peak V/a value, an intensity of peak/a value.

Clause 21: A system for determining a cognitive status of a test subject, comprising: an analytical component that is capable of receiving a sample obtained from the test subject, which analytical component is configured to determine three or more features from the sample obtained from the test subject using at least a thioflavin T (ThT) assay, a dynamic light scattering (DLS) assay, and a neurotoxicity assay to produce a test subject data set when the analytical component receives the sample; and, a controller that is operably connected, or connectable, at least to the analytical component, wherein the controller comprises, or is capable of accessing, computer readable media comprising non-transitory computer executable instructions which, when executed by at least one electronic processor, perform at least: using the test subject data set to classify the test subject as having a Parkinson's disease (PD) status of PD-NC (normal cognition) or PD-CI (cognitive impairment).

Clause 22: A system for determining a cognitive status of a test subject, the system comprising: a processor; and a memory communicatively coupled to the processor, the memory storing instructions which, when executed on the processor, perform operations comprising: passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and, outputting from the model a classification of the test subject as having a Parkinson's disease (PD) status of PD-NC (normal cognition) or PD-CI (cognitive impairment).

Clause 23: A computer readable media comprising non-transitory computer executable instructions which, when executed by at least one electronic processor, perform at least: receiving a test subject data set comprising three or more features determined from a sample obtained from the test subject using at least a thioflavin T (ThT) assay, a dynamic light scattering (DLS) assay, and a neurotoxicity assay; and, using the test subject data set to classify the test subject as having a Parkinson's disease (PD) status of PD-NC (normal cognition) or PD-CI (cognitive impairment).

Clause 24: A computer readable media comprising non-transitory computer executable instructions which, when executed by at least one electronic processor, perform at least: passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and, outputting from the model a classification of the test subject as having a Parkinson's disease (PD) status of PD-NC (normal cognition) or PD-CI (cognitive impairment).

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A method of determining a cognitive status of a test subject, the method comprising:
determining three or more features from a sample obtained from the test subject using at least a thioflavin T (ThT) assay, a dynamic light scattering (DLS) assay, and a neurotoxicity assay to produce a test subject data set; and,
using the test subject data set to classify the test subject as having a Parkinson's disease (PD) status of PD-NC (normal cognition) or PD-CI (cognitive impairment), thereby determining the cognitive status of the test subject.

2. The method of claim 1, comprising determining four or more, five or more, six or more, seven or more, eight or more, nine or more, ten or more, or more features from the sample.

3. The method of claim 1, wherein the features determined from the ThT assay comprise one or more of a ThT-mfi value, a ThT-tlag value, or a ThT-t50 value.

4. The method of claim 1, wherein the features determined from the DLS assay comprise one or more of a peak number value, a size of peak ½ value, an intensity of peak ½ value.

5. The method of claim 1, wherein when the cognitive status of the test subject is classified as PD-CI, the method further comprises classify the test subject as having PD-MCI (mild cognitive impairment) or PD-D (dementia).

6. The method of claim 1, further comprising repeating the method one or more times using samples obtained from the test subject at subsequent time points to evaluate the cognitive status of the test subject over a selected period of time.

7. The method of claim 1, further comprising predicting a probable disease progression outcome for the test subject based at least in part on the cognitive status of the test subject.

8. The method of claim 1, further comprising administering or discontinuing administering a therapy to the test subject based at least in part on the cognitive status of the test subject.

9. The method of claim 1, further comprising generating a therapy recommendation for the test subject based at least in part on the cognitive status of the test subject.

10. A computer-implemented method of determining a cognitive status of a test subject, the method comprising:
passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and,
outputting from the model a classification of the test subject as having a Parkinson's disease (PD) status of PD-NC (normal cognition) or PD-CI (cognitive impairment), thereby predicting the determining the cognitive status of the test subject.

11. The computer-implemented method of claim 10, wherein the model was created using the reference subject data sets produced by determining four or more, five or more, six or more, seven or more, eight or more, nine or more, ten or more, or more features from samples obtained from the reference subjects.

12. The computer-implemented method of claim 10, wherein the features determined from the ThT assay comprise one or more of a ThT-mfi value, a ThT-tlag value, or a ThT-t50 value.

13. The computer-implemented method of claim 10, wherein when the cognitive status of the test subject is classified as PD-CI, the model further outputs a classification of the test subject as having PD-MCI (mild cognitive impairment) or PD-D (dementia).

14. The computer-implemented method of claim 10, further comprising outputting from the model a prediction of a probable disease progression outcome for the test subject based at least in part on the cognitive status of the test subject.

15. The computer-implemented method of claim 10, further comprising outputting from the model a therapy recommendation for the test subject based at least in part on the cognitive status of the test subject.

16. The computer-implemented method of claim 10, wherein a trained electronic neural network comprises the model.

17. The computer-implemented method of claim 10, comprising determining three or more features from a sample obtained from the test subject using at least a thioflavin T (ThT) assay, a dynamic light scattering (DLS) assay, and a neurotoxicity assay to produce the test subject data set.

18. The computer-implemented of claim 17, comprising determining four or more, five or more, six or more, seven or more, eight or more, nine or more, ten or more, or more features from the sample obtained from the test subject.

19. The computer-implemented method of claim 17, wherein the features determined from the ThT assay comprise one or more of a ThT-mfi value, a ThT-tlag value, or a ThT-t50 value.

20. The computer-implemented method of claim 17, wherein the features determined from the DLS assay comprise one or more of a peak number value, a size of peak ½ value, an intensity of peak ½ value.

21. A system for determining a cognitive status of a test subject, comprising:
an analytical component that is capable of receiving a sample obtained from the test subject, which analytical component is configured to determine three or more features from the sample obtained from the test subject using at least a thioflavin T (ThT) assay, a dynamic light scattering (DLS) assay, and a neurotoxicity assay to produce a test subject data set when the analytical component receives the sample; and,
a controller that is operably connected, or connectable, at least to the analytical component, wherein the controller comprises, or is capable of accessing, computer readable media comprising non-transitory computer executable instructions which, when executed by at least one electronic processor, perform at least:
using the test subject data set to classify the test subject as having a Parkinson's disease (PD) status of PD-NC (normal cognition) or PD-CI (cognitive impairment).

22. A system for determining a cognitive status of a test subject, the system comprising:
a processor; and
a memory communicatively coupled to the processor, the memory storing instructions which, when executed on the processor, perform operations comprising:
passing at least one test subject data set through a model that relates test subject data sets to cognitive status of the test subjects, wherein the model was created using one or more reference subject data sets produced by determining three or more features from samples obtained from the reference subjects using at least a thioflavin T (ThT) assay, a dynamic light scattering (DLS) assay, and a neurotoxicity assay; and,
outputting from the model a classification of the test subject as having a Parkinson's disease (PD) status of PD-NC (normal cognition) or PD-CI (cognitive impairment).

23.-24. (canceled)
